(19) United States
(12) Patent Application Publication
(10) Pub. No.: US 2011/0129419 A1
(43) Pub. Date: Jun. 2, 2011

(54) IN VIVO IMAGING AGENTS

(75) Inventors: Anthony Eamon Storey, Lincs (GB); Julie Davis, Amersham (GB); Erik Arstad, London (GB); Benedicte Guilbert, Amersham (GB); Alessandra Gaeta, Amersham (GB)

(73) Assignees: GE HEALTHCARE LIMITED, Buckinghamshire (GB); HAMMERSMITH IMANET LIMITED, Greater London (GB)

(21) Appl. No.: 12/519,398

(22) PCT Filed: Dec. 19, 2007
(86) PCT No.: PCT/GB2007/004880

§ 371 Date: Dec. 8, 2009

(30) Foreign Application Priority Data
Dec. 21, 2006 (GB) 0625523.6

(51) Int. Cl. A61K 51/04; C07C 235/56; C07D 401/12; C07D 401/04; C07D 211/58; C07D 405/12; C07D 211/48; C07D 471/10

(52) U.S. Cl. 424/1.89; 564/174; 546/189; 546/201; 546/244; 546/207; 546/242; 544/365; 546/199; 544/231

(57) ABSTRACT

The present invention relates to the field of medical imaging, and in particular to imaging of disease states associated with the upregulation of the chemokine receptor 5 (CCR5). Imaging agents, precursors and methods are provided which are useful in imaging such disease states.

### TECHNICAL FIELD OF THE INVENTION

The present invention relates to the field of medical imaging, and in particular to in vivo imaging of disease states associated with the upregulation of a particular class of chemokine receptor (CCR). Compounds and methods are provided that are useful for imaging such disease states.

### DESCRIPTION OF RELATED ART

The chemokine system regulates the trafficking of immune cells to tissues and thus plays a central role in inflammation. The system is also involved in many other biological processes such as growth regulation, haematopoiesis and angiogenesis. In addition, chemokines are thought to play a central role in the central nervous system. Chemokines (chemotactic cytokines) are small secreted molecules characterised by 4 conserved cysteine residues forming two essential disulphide bonds (Cys1-Cys3; Cys2-Cys4). They can be briefly classified, based on the relative position of the two cysteine residues, as CC and CXC, which represent the two major classes. Chemokines act as chemical mediators, released either by invading immune cells or by resident cells locally at the site of inflammation.

Chemokines induce their biological effects through interaction with chemokine receptors (CCR). CCR are integral membrane proteins, formed of seven transmembrane α-helix domains linked by intracellular and extracellular loops, an extracellular N-terminus and a cytosolic C-terminus. They all share a common fold of three stranded antiparallel β-sheets covered on one face by a C-terminus α-helix and preceded by a disordered N-terminus. The dimerisation/oligomerisation process, essential for their functional properties, involves the N-terminus.

Expression of chemokine receptors (CCR) has been found to be perturbed in certain disease states where inflammation plays a role. For example, neuroinflammatory diseases such as multiple sclerosis (MS) [Rottman et al 2000 Eur. J. Immunol. 30 p 2372], Alzheimer's disease (AD) and Parkinson's disease (PD), [Xia & Hyman 1999 J. Neurovirology 5 p 32] and also other pathological inflammatory conditions such as atherosclerosis [Greaves & Channon 2002 Trends Immunol. 23(11) p 535], chronic obstructive pulmonary disorder (COPD), rheumatoid arthritis, osteoarthritis, allergic disease, HIV/AIDS, asthma and cancer.

One chemokine receptor that is particularly important in certain disease states is CCR5. It has been the subject of considerable therapeutic development as it is the chemokine receptor which the human immunodeficiency virus (HIV) uses to gain entry into macrophages and CCR5 expression is upregulated in chronic HIV infection. CCR5 has also received attention due to its involvement in the pathophysiology of various neuroinflammatory conditions such as MS, Alzheimer's disease and PD.

Chemokine receptor ligands have been reviewed by Gao and Metz [Chem. Rev., 103, 3733-52 (2003)], and Ribeiro and Horuk [Pharmacol. Ther., 107, 44-58 (2005)].

Targeting cytokine and chemokine receptors for nuclear medical imaging has been described as a challenge [Signore et al, Eur. J. Nucl. Med. Mol. Imaging, 30(1), 149-165 (2003)]. Signore et al reported that the main approach known to target chemokine receptors was radiolabelled interleukin-8 (IL-8).

WO 02/36581 teaches radiopharmaceuticals that bind to the CCR1 receptor and that are able to pass through the blood-brain barrier (BBB). These radiopharmaceuticals are taught as useful in diagnosing Alzheimer's disease.

WO 2006/102395 teaches targeting of imaging moieties (referred to therein as “imaging agents”) to atherosclerotic plaques. The ligand RANTES, which binds to the CCR5 receptor, is taught as one of a number of targeting moieties suitable for the delivery of an imaging moiety to atherosclerotic lesions when linked thereto. The imaging moieties taught include those suitable for a range of in vivo imaging modalities, e.g. single photon emission tomography (SPECT), magnetic resonance imaging (MRI) and positron emission tomography (PET).

The ability to image conditions where CCR5 is specifically implicated, especially neuroinflammation, may represent an important tool for early diagnosis of different acute and chronic pathological conditions and to support therapeutic approaches and strategies. There is therefore a need for imaging agents which image CCR5, and in particular those that can cross the BBB.

### SUMMARY OF THE INVENTION

The present invention relates to in vivo imaging and in particular to novel imaging agents suitable for use in in vivo imaging of the chemokine receptor 5 (CCR5). The invention also provides a method for the preparation of the imaging agents of the invention as well as pharmaceutical compositions comprising them. For the facile preparation of the pharmaceutical compounds, kits are provided. In addition, the invention provides methods for the use of the imaging agents and pharmaceutical compositions of the invention.

### DETAILED DESCRIPTION OF THE INVENTION

In a first aspect, the present invention provides an imaging agent which comprises a synthetic compound having affinity for chemokine receptor 5 (CCR5) and having a molecular weight of 3000 Daltons or less, labelled with at least one imaging moiety, wherein following administration of said compound to the mammalian body in vivo, the imaging moiety can be detected externally in a non-invasive manner and said imaging moiety is chosen from:
(i) a gamma-emitting radioactive halogen; or
(ii) a positron-emitting radioactive non-metal.

A compound having “affinity for CCR5” is defined in the present invention as that which inhibits binding of MIP-1β CCR5-expressing CHO cells with IC₅₀ values of between 0.1 nM to 10 nM, where MIP-1β is Macrophage Inflammatory Protein 1β (ligand of CCR5) [Samson et al., J. Biol. Chem., 272, 24934-41 (1997)]. See also Example 4. The CCR5 compounds of the present invention are also preferably selective for CCR5 over other chemokine receptors (such as CCR1 or CCR3). Such selective inhibitors suitably exhibit a greater potency for CCR5 over CCR1, defined by Ki, of a factor of at least 50, preferably at least 100, most preferably at least 500.

The synthetic compound is preferably a non-peptide. By the term “non-peptide” is meant a compound which does not comprise any peptide bonds, i.e. an amide bond between two amino acid residues. The synthetic compound having affinity for chemokine receptor 5 (CCR5) preferably has a molecular weight of 1000 Daltons or less, and most preferably 600 Daltons or less. The synthetic compound preferably comprises 2 to 6, most preferably 2 to 5 nitrogen (N) atoms. Said N atoms are present as part of amide; amine; or 5- or 6-membered nitrogen-containing heteroaryl ring functional groups. The heteroaryl ring can have 1 or 2 N heteroatoms. When an amine is present, it is suitably either open chain or as part of a 5- or 6-membered saturated aliphatic ring. Preferred such cyclic amines are piperidine, piperazine or morpholine. When an amide is present, it is suitably open chain, i.e. does not comprise a lactam. Preferred such amides are benzamides or acyl derivatives of aniline, benzylamine or aminopiperidine residues. The synthetic compound also preferably comprises 1 to 3 phenyl rings, most preferably 1 or 2 phenyl rings. The CCR5 pharmacophore preferably comprises two hydrogen bond acceptors and three hydrophobic interactions; in particular it has a basic amine located 5-7 Å from a phenyl ring.

The term “labelled with” means that either a functional group comprises the imaging moiety, or the imaging moiety is attached as an additional species. When a functional group comprises the imaging moiety, this means that the ‘imaging moiety’ forms part of the chemical structure, and is a radioactive isotope present at a level significantly above the natural abundance level of said isotope. Such elevated or enriched levels of isotope are suitably at least 5 times, preferably at least 10 times, most preferably at least 20 times; and ideally either at least 50 times the natural abundance level of the isotope in question, or present at a level where the level of enrichment of the isotope in question is 90 to 100%. Examples of such functional groups include CH₃ groups with elevated levels of ¹¹C, and fluoroalkyl groups with elevated levels of ¹⁸F, such that the imaging moiety is the isotopically labelled ¹¹C or ¹⁸F atom within the chemical structure. The radioisotopes ³H and ¹⁴C are not suitable imaging moieties.

When the imaging moiety is a gamma-emitting radioactive halogen, the radiohalogen is suitably chosen from ¹²³I, ¹³¹I or ⁷⁷Br. A preferred gamma-emitting radioactive halogen is ¹²³I. When the imaging moiety is a positron-emitting radioactive non-metal, the imaging agent is suitable for positron emission tomography (PET). Suitable such positron emitters include: ¹¹C, ¹³N, ¹⁷F, ¹⁸F, ⁷⁵Br, ⁷⁶Br or ¹²⁴I. Preferred positron-emitting radioactive non-metals are ¹¹C, ¹³N, ¹²⁴I and ¹⁸F, especially ¹¹C and ¹⁸F, most especially ¹⁸F.

The imaging moiety is preferably a positron-emitting radioactive non-metal. The use of a PET imaging moiety has certain technical advantages, including:
(i) the development of PET/CT cameras allowing easy co-registration of functional (PET) and anatomical (CT) images for improved diagnostic information;
(ii) the facility to quantify PET images to allow accurate assessment for staging and therapy monitoring;
(iii) increased sensitivity to allow visualisation of smaller target tissues.

In one embodiment, the imaging agent comprises a synthetic compound of Formula I:

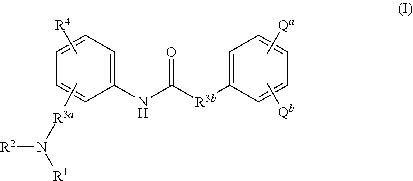

wherein:
R¹ and R² are independently C₁₋₆ alkyl, or C₁₋₆ haloalkyl;
R^{3a} and R^{3b} are independently represent a bond, or a linker group selected from C₁₋₅ alkylene, —O—[C₁₋₄ alkylene]- or —[C₁₋₂ alkylene]-O—[C₁₋₂ alkylene]-;
R⁴ is selected from H, C₁₋₆ alkyl or C₁₋₆ alkoxy; and,
Q^{a} and Q^{b} are independently an A³ group or -(A²)ₙ-R⁵;
wherein A² is selected from —O—, —OCH₂—, —CH₂O—, CH₂, C═O, S═O, SO₂, —NH(CO)— or —CO(NH)—, R⁵ is a phenyl group with 0-3 substituents which are A³ groups, and n is an integer of value 0 to 3;
wherein -A³ is H, C₁₋₆ alkyl, OH or Hal.

Preferred compounds of Formula I are as follows:

R¹ and R² are independently selected from methyl, ethyl, 1-methylethyl, fluoromethyl, 2-fluoroethyl, 3-fluoropropyl or 1-fluoromethylethyl;

R^{3a} and R^{3b} are independently C₁₋₃ alkylene or C₁₋₃ alkoxy;

R⁴ is H or a C₁₋₃ alkyl;

Q is 3-phenoxy, 4-phenoxy, 4-(3-hydroxyphenoxy), 4-(4-methylphenyl)sulfonyl, 4-(4-chlorophenyl)sulfonyl, 4-(2,4-dichlorophenyl)sulfonyl, 4-(4-chlorophenoxy), 4-methylphenylamino, 4-phenylamino, 4-phenylthio, 4-phenylsulfonyl, 4-benzoyl, 4-(4-iodophenoxy), 3-(4-iodophenoxy), 4-(4-fluorophenoxy), 3-(4-fluorophenoxy), or 3-(4-fluoroethyl)phenoxy.

Preferred imaging agents which comprise compounds of Formula I are of Formula Ia:

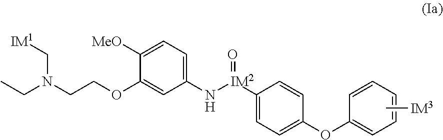

wherein:
IM¹ and IM³ are independently H or an imaging moiety;
IM² is C or the imaging moiety ¹¹C;
with the proviso that at least one of IM¹⁻³ is an imaging moiety.

Preferred compounds of Formula Ia are as follows:

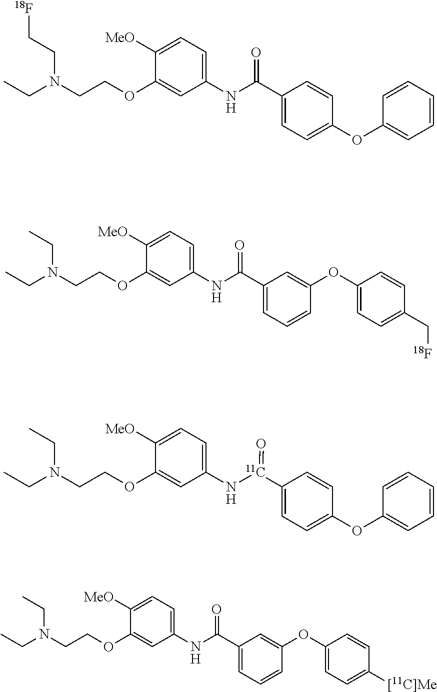

An alternative preferred compound of Formula I is a compound of Formula Ib:

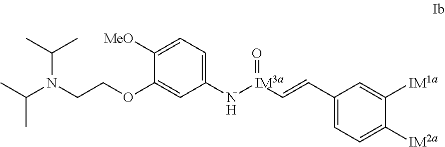

wherein
IM^{1a} and IM^{2a} are independently H, Hal, or an imaging moiety;
IM^{3a} is C or the imaging moiety ¹¹C;
with the proviso that at least one of IM¹⁻³ is an imaging moiety.

In a further embodiment, the imaging agent comprises a synthetic compound of Formula II:

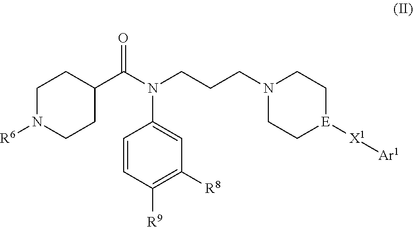

wherein:
R⁶ is acyl, fluoroacyl or methylsulfonyl;
R⁸-R⁹ are independently selected from H, C₁₋₃ alkyl, OH or Hal.
E is N or CH;
when E is N, X¹ is —CH₂— and when E is CH, X¹ is —CH₂— or —O—;
Ar¹ is a 6-membered aryl ring having 0-2 N heteroatoms, and substituted with 0 to 3 R⁷ groups;
   each R⁷ is independently chosen from C₁₋₃ alkyl, OH, Hal, NO₂, NH₂, CO₂H, C₁₋₆ alkoxy, C₁₋₆ amino, C₁₋₆ amido, —O(CH₂CH₂O)ₓX² or —NH(CH₂CH₂O)ₓX² where x is an integer of value 0 to 4, and X² is H or CH₃.

In Formula II, R⁶ is preferably acetyl; R⁸-R⁹ are preferably selected from H, CH₃, OH, Cl, F and I; and Ar¹ preferably comprises a phenyl or pyridine ring, most preferably a phenyl ring. Preferably, the Ar¹ ring is unsubstituted or substituted with one R⁷ group. When present, R⁷ is preferably chosen from: —OH, —NHCH₃, F, —O(CH₂CH₂O)ₓX² or —NH(CH₂CH₂O)ₓX². X² is preferably H.

Preferred imaging agents which comprise compounds of Formula II are of Formula IIa:

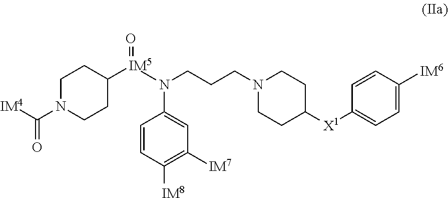

wherein:
IM⁴ is independently H, CH₃ or an imaging moiety
IM⁶, IM⁷ and IM⁸ are independently H or an imaging moiety;
IM⁵ is C or the imaging moiety ¹¹C;
with the proviso that at least one of IM⁴⁻⁸ is an imaging moiety; and,
X¹ is as defined above for Formula II.

Preferred compounds of Formula IIa are:

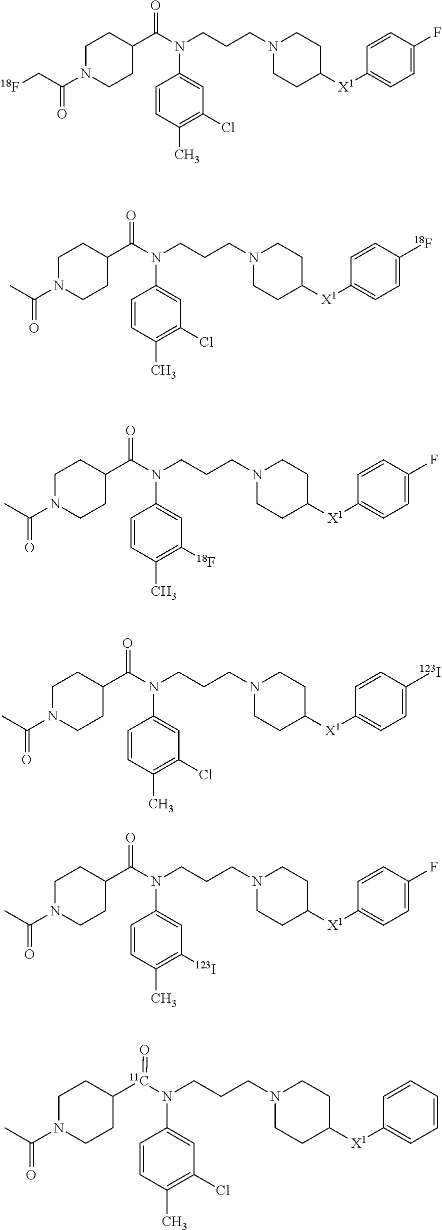

wherein X¹ is as defined above for Formula II.

In a further embodiment, the imaging agent comprises a synthetic compound of Formula III:

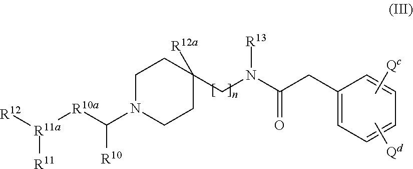

wherein:
R¹⁰ is H or C₁₋₃ alkyl;
R^{10a} is CH₂ or a phenylene group with 0-2 substituents selected from C₁₋₃ alkyl, C₁₋₃ haloalkyl, or Hal;
R¹¹ is H or a phenyl group with 0-3 substituents independently selected from OH, Hal, C₁₋₆ alkyl, C₁₋₆ alkoxyalkyl, C₁₋₆ fluoroalkyl or nitrile;
R^{11a} is selected from CH, C₁₋₃ alkylene, and —O—C₁₋₃ alkylene;
R¹² is a phenyl group with 0-3 substituents selected from C₁₋₃ alkyl, C₁₋₃ haloalkyl, Hal, C₁₋₃ alkylsulfonyl, or R¹² is —NHC═O—R^{x}R^{y} wherein:
R^{x} is selected from oxygen and (CH₂)ₚ wherein p is an integer of value 0 to 3; and,
R^{y} is a six-membered ring with 0-3 heteroatoms selected from O, N and S;
R^{12a} is H or OH;
R¹³ is H, C₁₋₃ alkyl or a C₁₋₃ haloalkyl; and,
Q^{c} and Q^{d} are independently substituents selected from H, Hal, C₁₋₃ alkyl, and C₁₋₃ alkyl sulfonyl.

Preferred compounds of Formula III have:
R¹⁰═H;
R^{10a}═CH₂;
R¹¹=3-fluorophenyl, 4-fluorophenyl, 3-chlorophenyl, 3,5-difluorophenyl, 3-fluoro, 4-chloro-phenyl, 3-hydroxy,4-iodophenyl, 4-iodophenyl;
R^{11a}═CH;
R¹²=a phenyl group with 1 or 2 substituents selected from C₁₋₃ alkyl, C₁₋₃ haloalkyl, Hal, C₁₋₃ alkylsulfonyl or R¹² is NHC═O—R^{x}R^{y} wherein:
   R^{x} is oxygen; and,
   R^{y} is cyclohexyl, dihydropyran or tetrahydropyran;
R^{12a}═H;
R¹³=ethyl, fluoroethyl, propyl, or fluoropropyl; and,
Q^{c} is H and Q^{d} is 4-C₁₋₃ alkyl or 4-C₁₋₃ alkyl sulfonyl.

Alternatively preferably, for compounds of Formula III:
R¹⁰═H or CH₃;
R^{10a}=a phenylene group with a Hal substituent;
R¹¹═H;
R^{11a}═C₁₋₃ alkoxy;
R¹²=a phenyl group with 1 or 2 substituents selected from C₁₋₃ alkyl, C₁₋₃ haloalkyl, Hal, C₁₋₃ alkylsulfonyl or R¹² is NHC═O—R^{x}R^{y} wherein:
   R^{x} is oxygen; and,
   R^{y} is cyclohexyl, dihydropyran or tetrahydropyran;
R^{12a}═OH;
R¹³=ethyl, fluoroethyl, propyl, or fluoropropyl; and,
Q^{c} is H and Q^{d} is 4-C₁₋₃ alkyl or 4-C₁₋₃ alkyl sulfonyl.

Preferred imaging agents which comprise compounds of Formula III are of Formulae IIIa-IIIc:

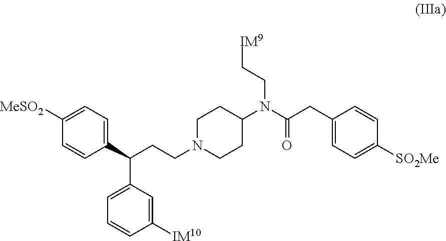

wherein:
IM⁹ and IM¹⁰ are independently H or an imaging moiety;
with the proviso that at least one of IM⁹⁻¹⁰ is an imaging moiety.

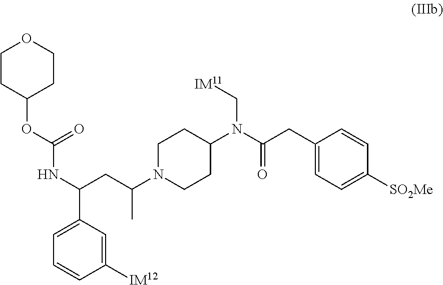

wherein:
IM¹¹ and IM¹² are independently H, CH₃ or an imaging moiety;
with the proviso that at least one of IM¹¹⁻¹² is an imaging moiety.

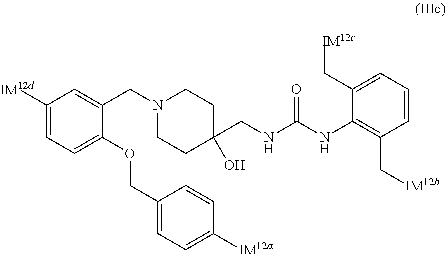

wherein:
IM^{12a}-IM^{12d} are independently H, CH₃ or an imaging moiety;
with the proviso that at least one of IM^{12a}-IM^{12d} is an imaging moiety.

Preferred imaging agents of Formula IIIa are selected from:

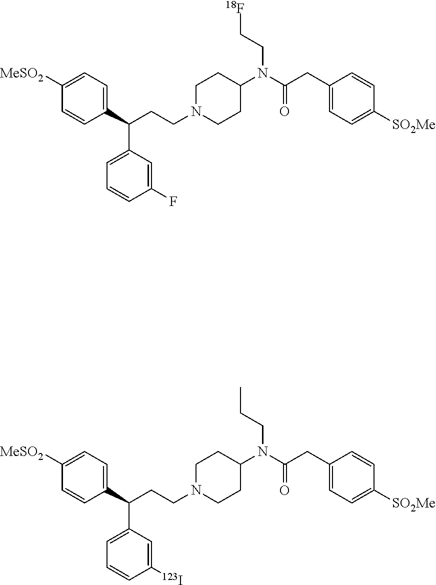

Preferred imaging agents of Formula IIIb are selected from:

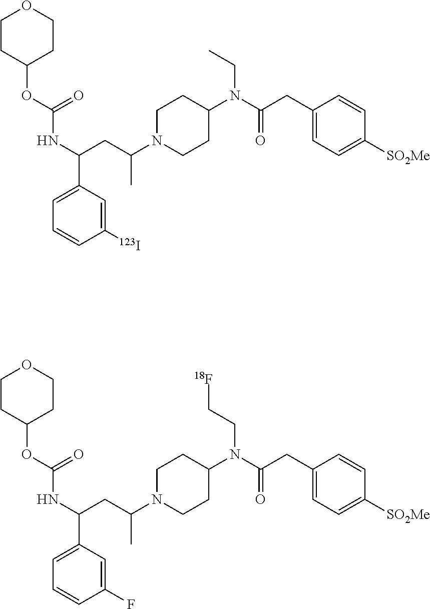

In a further embodiment, the imaging agent comprises a synthetic compound of Formula IV:

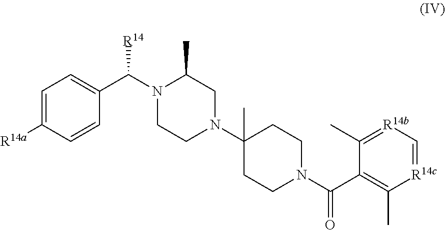

wherein:

R¹⁴ is H, C₁₋₆ alkyl, C₁₋₆ fluoroalkyl, C₁₋₆ alkoxy, or a phenyl or benzyl group optionally substituted with an A⁴ group;
wherein A⁴ is C₁₋₆ alkyl, C₁₋₆ alkoxy or Hal;

R^{14a} is selected from Hal or C₁₋₃ haloalkyl; and,

R^{14b} and R^{14c} are independently selected from CH₂ or N.

Preferably in Formula IV:

R¹⁴ is C¹⁻³ fluoroalkyl or halophenyl;

R^{14a} is C₁₋₃ haloalkyl; and,

R^{14b} and R^{14c} are both N.

Alternatively preferably in Formula IV:

R¹⁴ is C₁₋₃ alkyl;

R^{14a} is Hal; and,

R^{14b} and R^{14c} are both CH₂.

Preferred imaging agents which comprise compounds of Formula IV are of Formula IVa or Formula IVb:

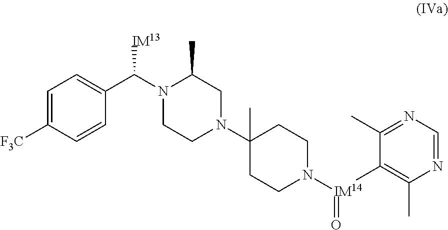

wherein:
IM¹³ is independently CH₃ or an imaging moiety;
IM¹⁴ is independently C or the imaging moiety ¹¹C;
with the proviso that at least one of IM¹³⁻¹⁴ is an imaging moiety.

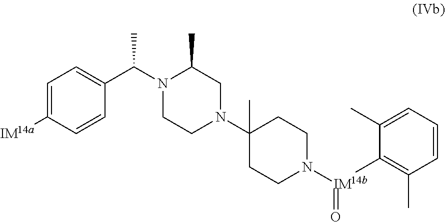

wherein:
IM^{14a} is independently CH₃ or an imaging moiety;
IM^{14b} is independently C or the imaging moiety ¹¹C;
with the proviso that at least one of IM^{14a-14b} is an imaging moiety.

Preferred imaging agents of Formula IVa are selected from:

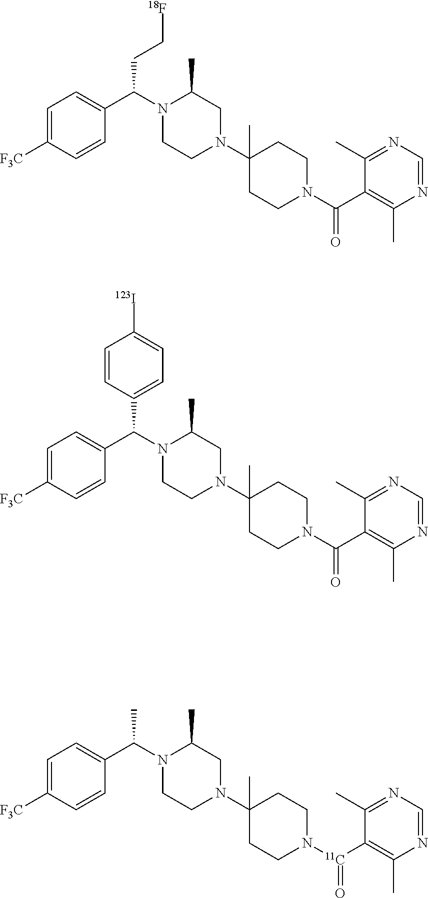

Preferred imaging agents of Formula IVb are selected from:

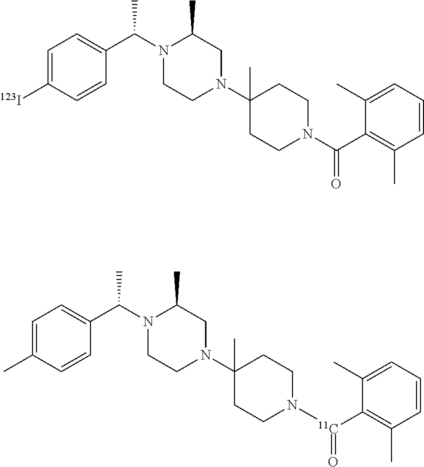

In a further embodiment, the imaging agent comprises a synthetic compound of Formula V:

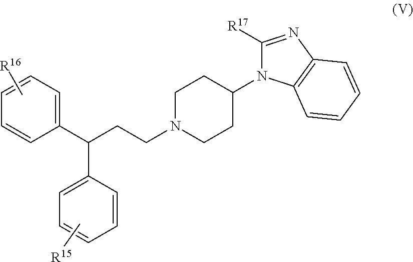

wherein:
R¹⁵ and R¹⁶ are independently H, OH, C₁₋₃ alkyl or Hal; and,
R¹⁷ is H, C₁₋₆ alkyl, or C₁₋₆ haloalkyl.

Preferred compounds of Formula V are those wherein:
R¹⁵ and R¹⁶ are independently H or Hal; and,
R¹⁷ is C₁₋₃ fluoroalkyl.

Preferred imaging agents which comprise compounds of Formula V are of Formula Va:

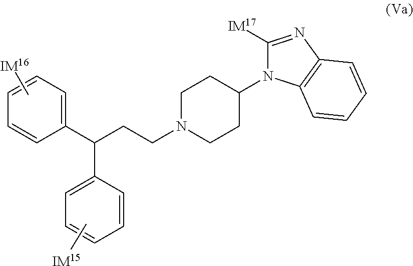

wherein:
IM¹⁵ to IM¹⁷ are independently H or an imaging moiety;
with the proviso that at least one of IM¹⁵⁻¹⁷ is an imaging moiety.

Preferred imaging agents of Formula Va are selected from:

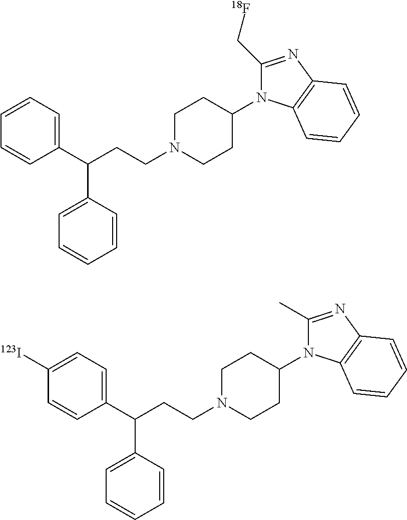

In a further embodiment, the imaging agent comprises a synthetic compound of Formula VI:

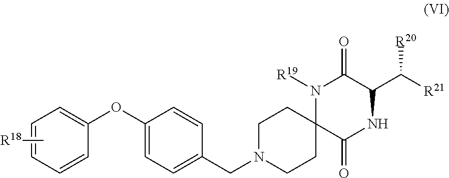

wherein:

R¹⁸ is H or Hal;

R¹⁹ is C₁₋₆ alkyl or C₁₋₆ haloalkyl;

R³⁹ H, OH, or Hal; and,

R²¹ is C₁₋₆ alkyl, C₁₋₆ cycloalkyl, or C₁₋₆ haloalkyl.

Examples of preferred imaging agents of Formula VI are as follows:

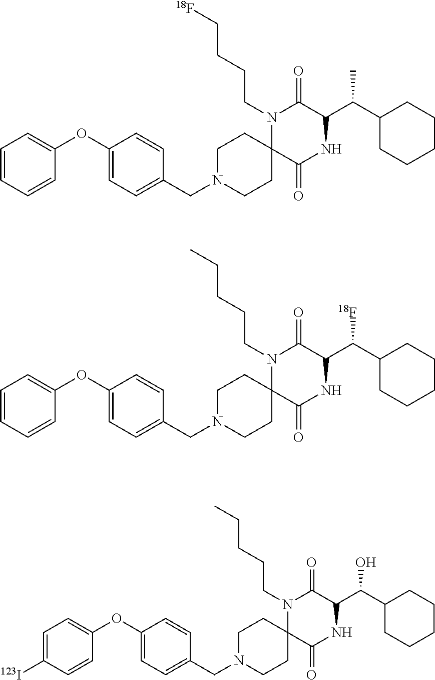

The synthetic compound having affinity for chemokine receptor 5 (CCR5) can be obtained as follows:

Formula I—WO 00/06146, Shiraishi et al [J. Med. Chem. 43 pp 2049-63 (2000)].

Formula II—Piperidine-4-carboxamide derivatives, Imamura et al, [Bioorg. Med. Chem. 13 p. 397-416 (2005), and J. Med. Chem. 49 pp 2784-93 (2006)].

Formula III—diphenylpropylpiperidine derivatives, Cumming et al [Bioorg. Med. Chem. Lett., 16 p 3533-3536 (2006)], and Shou-Fu Lu et al. Bioorg. Med. Chem. Lett., 2007, 17, 1883-1887.

Formula IV—piperazine-based derivatives, Tagat et al [J. Med. Chem., 47, 2405-8 (2004)]; and Tagat et al [J. Med. Chem 44, 3343-6 (2001)]

Formula V—Wood and Armour [Prog. Med. Chem., 43, 239-271(2005)]

Formula VI—Mitsuya et al [J. Med. Chem. 49 pp 4140-52 (2006), and Bioorg. Med. Chem. Lett. 17 pp 727-31 (2007)]

The imaging agents of the first aspect are suitably prepared by reaction with a precursor, as described in the second aspect below.

In a second aspect, the present invention provides a method for the preparation of the imaging agent of the first aspect, which comprises reaction of:
(i) a non-radioactive precursor; and,
(ii) a suitable source of the imaging moiety of the first aspect,

wherein said precursor is a derivative of the synthetic compound of the first aspect, and said derivative comprises a substituent Y¹ which is capable of reaction with said suitable source of the imaging moiety to give the desired imaging agent.

The “precursor” suitably comprises a non-radioactive derivative of the synthetic compound, which is designed so that chemical reaction with a convenient chemical form of the desired non-metallic radioisotope can be conducted in the minimum number of steps (ideally a single step), and without the need for significant purification (ideally no further purification) to give the desired radioactive product. Such precursors are synthetic and can conveniently be obtained in good chemical purity. The “precursor” may optionally comprise a protecting group (P^{GP}) for certain functional groups of the synthetic CCR5 compound. Suitable precursors are described by Bolton, J. Lab. Comp. Radiopharm., 45, 485-528 (2002).

By the term “protecting group” (P^{GP}) is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tert-butyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999).

Preferred precursors are those wherein Y¹ comprises a derivative which either undergoes direct electrophilic or nucleophilic halogenation; undergoes facile alkylation with a labelled alkylating agent chosen from an alkyl or fluoroalkyl halide, tosylate, triflate (i.e. trifluoromethanesulphonate), mesylate, maleimide or a labelled N-haloacetyl moiety; alkylates thiol moieties to form thioether linkages; or undergoes condensation with a labelled active ester, aldehyde or ketone. Examples of the first category are:
(a) organometallic derivatives such as a trialkylstannane (e.g. trimethylstannyl or tributylstannyl), or a trialkylsilane (e.g. trimethylsilyl);
(b) a non-radioactive alkyl iodide or alkyl bromide for halogen exchange and alkyl tosylate, mesylate or triflate for nucleophilic halogenation;
(c) aromatic rings activated towards electrophilic halogenation (e.g. phenols) and aromatic rings activated towards nucleophilic halogenation (e.g. aryl is iodonium, aryl diazonium, aryl trialkylammonium salts or nitroaryl derivatives).

Preferred derivatives which undergo facile alkylation are alcohols, phenols, amine or thiol groups, especially thiols and sterically-unhindered primary or secondary amines.

Preferred derivatives which alkylate thiol-containing radioisotope reactants are maleimide derivatives or N-haloacety) groups. Preferred examples of the latter are N-chloroacetyl and N-bromoacetyl derivatives.

Preferred derivatives which undergo condensation with a labelled active ester moiety are amines, especially sterically-unhindered primary or secondary amines.

Preferred derivatives which undergo condensation with a labelled aldehyde or ketone are aminooxy and hydrazides groups, especially aminooxy derivatives.

The “precursor” may optionally be supplied covalently attached to a solid support matrix. In that way, the desired imaging agent product forms in solution, whereas starting materials and impurities remain bound to the solid phase. Precursors for solid phase electrophilic fluorination with ¹⁸F-fluoride are described in WO 03/002489. Precursors for solid phase nucleophilic fluorination with ¹⁸F-fluoride are described in WO 03/002157. The solid support-bound precursor may therefore be provided as a kit cartridge which can be plugged into a suitably adapted automated synthesizer. The cartridge may contain, apart from the solid support-bound precursor, a column to remove unwanted fluoride ion, and an appropriate vessel connected so as to allow the reaction mixture to be evaporated and allow the product to be formulated as required. The reagents and solvents and other consumables required for the synthesis may also be included together with a compact disc carrying the software which allows the synthesiser to be operated in a way so as to meet the customer requirements for radioactive concentration, volumes, time of delivery etc. Conveniently, all components of the kit are disposable to minimise the possibility of contamination between runs and will be sterile and quality assured.

When the imaging moiety comprises a radioactive iodine isotope, Y¹ suitably comprises: a non-radioactive precursor halogen atom such as an aryl iodide or bromide (to permit radioiodine exchange); an activated precursor aryl ring (e.g. phenol or aniline groups); an imidazole ring; an indole ring; an organometallic precursor compound (e.g. trialkyltin or trialkylsilyl); or an organic precursor such as triazenes or a good leaving group for nucleophilic substitution such as an iodonium salt. Methods of introducing radioactive halogens (including ¹²³I and ¹⁸F) are described by Bolton [J. Lab. Comp. Radiopharm., 45, 485-528 (2002)]. Examples of suitable precursor aryl groups to which radioactive halogens, especially iodine can be attached are given below:

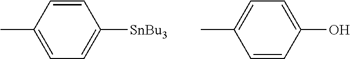

Both contain substituents which permit facile radioiodine substitution onto the aromatic ring. Alternative substituents containing radioactive iodine can be synthesised by direct iodination via radiohalogen exchange, e.g.

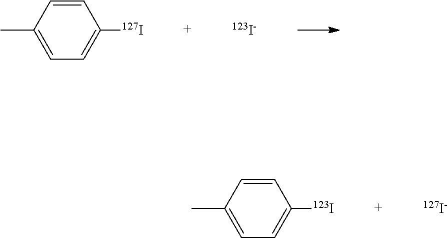

For radioactive isotopes of iodine, the radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine. An iodine atom bound to an activated aryl ring like phenol has also, under certain circumstances, been observed to have limited in vivo stability.

When the imaging moiety comprises a radioactive isotope of fluorine the radiofluorine atom may form part of a fluoroalkyl or fluoroalkoxy group, since alkyl fluorides are resistant to in vivo metabolism. For radioactive isotopes of fluorine (e.g. ¹⁸F), the radiohalogenation may be carried out via direct labelling using the reaction of ¹⁸F-fluoride with a suitable precursor having a good leaving group, such as an alkyl bromide, alkyl mesylate or alkyl tosylate. Alternatively, the radiofluorine atom may be attached via a direct covalent bond to an aromatic ring such as a benzene ring. For such aryl systems, the precursor suitably comprises an activated nitroaryl ring, an aryl diazonium salt, or an aryl trialkylammonium salt. Direct radiofluorination can, however, be detrimental to sensitive functional groups since these nucleophilic reactions are carried out with anhydrous [¹⁸F]fluoride ion in polar aprotic solvents under strong basic conditions.

When the synthetic compound has alkali-sensitive functional groups, or other functionality unsuitable for direct radiohalogenation, an indirect radiohalogenation method is preferred. Thus, when the imaging moiety comprises a radioactive halogen, such as ¹²³I and ¹⁸F, Y¹ preferably comprises a functional group that will react selectively with a radiolabelled synthon and thus upon conjugation gives the desired imaging agent product. By the term “radiolabelled synthon” is meant a small, synthetic organic molecule which is:
(i) already radiolabelled such that the radiolabel is bound to the synthon in a stable manner;
(ii) comprises a functional group designed to react selectively and specifically with a corresponding functional group which is part of the desired compound to be radiolabelled. This approach gives better opportunities to generate imaging agents with improved in vivo stability of the radiolabel relative to direct radiolabelling approaches.

A synthon approach also allows greater flexibility in the conditions used for the introduction of the imaging moiety.

¹⁸F-can also be introduced-by N-alkylation of amine precursors with alkylating agent synthons such as ¹⁸F(CH₂)₃OMs (where Ms is mesylate) to give N—(CH₂)₃¹⁸F, O-alkylation of hydroxyl groups with ¹⁸F(CH₂)₃OMs,¹⁸F(CH₂)₃OTs or ¹⁸F(CH₂)₃Br or S-alkylation of thiol groups with ¹⁸F(CH₂)₃OMs or ¹⁸F(CH₂)₃Br. ¹⁸F can also be introduced by alkylation of N-haloacetyl groups with a ¹⁸F(CH₂)₃OH reactant, to give —NH(CO)CH₂O(CH₂)₃¹⁸F derivatives or with a ¹⁸F(CH₂)₃SH reactant, to give —NH(CO)CH₂S(CH₂)₃¹⁸F derivatives. ¹⁸F can also be introduced by reaction of maleimide-containing precursors with ¹⁸F(CH₂)₃SH. For aryl systems, ¹⁸F-fluoride nucleophilic displacement from an aryl diazonium salt, an aryl nitro compound or an aryl quaternary ammonium salt are suitable routes to aryl-¹⁸F labelled synthons useful for conjugation to precursors of the imaging agent.

Precursors wherein Y¹ comprises a primary amine group can also be labelled with ¹⁸F by reductive amination using ¹⁸F—C₆H₄—CHO as taught by Kahn et al [J. Lab. Comp. Radiopharm. 45, 1045-1053 (2002)] and Borch et al [J. Am. Chem. Soc. 93, 2897 (1971)]. This approach can also usefully be applied to aryl primary amines, such as compounds comprising phenyl-NH₂ or phenyl-CH₂NH₂ groups.

An especially preferred method for base-sensitive precursors is when Y¹ comprises an aminooxy group of formula —NH(C═O)CH₂—O—NH₂ which is condensed with ¹⁸F—C₆H₄—CHO under acidic conditions (e.g. pH 2 to 4). Further details of synthetic routes to ¹⁸F-labelled derivatives are described by Bolton, J. Lab. Comp. Radiopharm., 45, 485-528 (2002).

The precursor is preferably in sterile, apyrogenic form. Methods for maintaining 3 0 sterility are described in the third aspect below.

Examples of precursors suitable for the generation of imaging agents of the present invention are those where Y¹ comprises an amine group which is condensed with the synthon N-succinimidyl 4-[¹²³I]iodobenzoate at pH 7.5-8.5 to give amide bond linked products.

Preferred precursors comprising the compounds of Formula I to Formula V are of Formula Ip to Vp respectively:

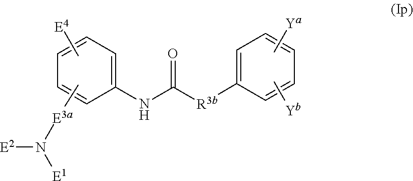

wherein at least one of E¹-E⁴ and Y^{a}-Y^{b} comprises Y¹, and the remaining E¹-E⁴ and Y^{a}-Y^{b} groups are R¹-R⁴ and Q^{a}-Q^{b} groups respectively of Formula I.

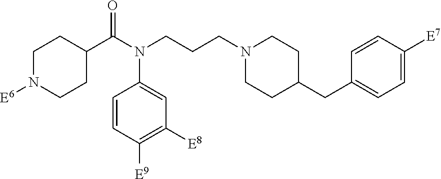

wherein at least one of E⁶-E⁹ comprises Y¹, and the remaining E⁶-E⁹ groups are R⁶-R⁹ groups respectively of Formula II.

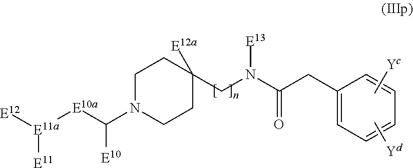

wherein at least one of E¹⁰, E¹¹, E¹², or E¹³ comprises Y¹ and the remaining E¹⁰, E¹¹, E¹², or E¹³ groups are R¹⁰-R¹³ groups respectively of Formula III;
E^{11a}-E^{12a} are as defined for E^{11a}-E^{12a} of Formula III; and,
Y^{c} and Y^{d} are as defined for Q^{c} and Q^{d} of Formula III.

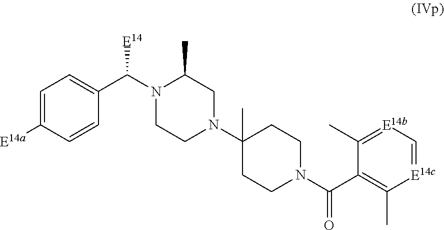

wherein E¹⁴ is an R¹⁴ group of Formula IV which comprises Y¹; and,
E^{14a}-E^{14c} are as defined for R^{14a}-R^{14c} of Formula IV.

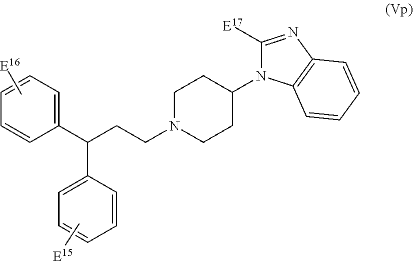

wherein at least one of E¹⁵-E¹⁷ comprises Y¹ and the remaining E¹⁵-E¹⁷ groups are R¹⁵-R¹⁷ respectively of Formula V.

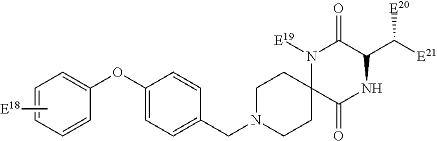

wherein at least one of E¹⁸-E²⁰ comprises Y¹ and the remaining E¹⁸-E²⁰ groups are R¹⁸-R²⁰ respectively of Formula VI; and,
E²¹ is R²¹ as defined for Formula VI.

In a third aspect, the present invention provides a pharmaceutical composition which comprises the imaging agent of the first aspect together with a biocompatible carrier, in a form suitable for mammalian administration.

The “biocompatible carrier” is a fluid, especially a liquid, in which the imaging agent can be suspended or dissolved, such that the composition is physiologically tolerable, i.e. can be administered to the mammalian body without toxicity or undue discomfort. The biocompatible carrier is suitably On injectable carrier liquid such as sterile, pyrogen-free water for injection; an aqueous solution such as saline (which may advantageously be balanced so that the final product for injection is isotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection or isotonic saline.

Such radioactive pharmaceutical compositions (i.e. radiopharmaceutical compositions) are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pre-filled syringe may optionally be provided with a syringe shield to protect the operator from radioactive dose. Suitable such radiopharmaceutical syringe shields are known in the art and preferably comprise either lead or tungsten.

The radiopharmaceutical compositions may be prepared from kits, as is described in the fourth aspect below. Alternatively, the radiopharmaceuticals may be prepared under aseptic manufacture conditions to give the desired sterile product. The radiopharmaceuticals may also be prepared under non-sterile conditions, followed by terminal sterilisation using e.g. gamma-irradiation, autoclaving, dry heat or chemical treatment (e.g. with ethylene oxide).

In a fourth aspect, the present invention provides a kit for the preparation of the pharmaceutical composition of the third aspect, which kit comprises the precursor of the second aspect: Such kits comprise the “precursor” of the second-aspect, preferably in sterile non-pyrogenic form, so that reaction with a sterile source of the radioisotopic imaging moiety gives the desired radiopharmaceutical with the minimum number of manipulations. Such considerations are particularly important when the radioisotope has a relatively short half-life, and for ease of handling and hence reduced radiation dose for the radiopharmacist. Hence, the reaction medium for reconstitution of such kits is preferably a “biocompatible carrier” as defined above, and is most preferably aqueous.

Suitable kit containers comprise a sealed container which permits maintenance of sterile integrity and/or radioactive safety, plus optionally an inert headspace gas (e.g. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

The non-radioactive kits may optionally further comprise additional components such as a radioprotectant, antimicrobial preservative, pH-adjusting agent or filler. By the term “radioprotectant” is meant a compound which inhibits degradation reactions, such as redox processes, by trapping highly-reactive free radicals, such as oxygen- containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation. By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the radiopharmaceutical composition post-reconstitution, i.e. in the radioactive diagnostic product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the conjugate is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

Preferred aspects of the “precursor” when employed in the kit are as described for the second aspect above. The precursors for use in the kit may be employed under aseptic manufacture conditions to give the desired sterile, non-pyrogenic material. The precursors may also be employed under non-sterile conditions, followed by terminal sterilisation using e.g. gamma-irradiation, autoclaving, dry heat or chemical treatment (e.g. with ethylene oxide). Preferably, the precursors are employed in sterile, non-pyrogenic form. Most preferably the sterile, non-pyrogenic precursors are employed in the sealed container as described above.

In a fifth aspect, the present invention provides a method for the in vivo diagnosis or imaging in a subject of a CCR5 condition, comprising administration of the pharmaceutical composition of the third aspect. By the term “CCR5 condition” is meant a disease state of the mammalian, especially human, body where CCR5 expression is upregulated or downregulated. Preferably, the CCR5 expression is upregulated since that should give better signal-to-background in diagnostic imaging in vivo. CCR5 expression is upregulated in chronic HIV infection. CCR5 conditions also include various pathological inflammatory conditions as well as neuroinflammatory conditions. Pathological inflammatory conditions include: atherosclerosis, chronic obstructive pulmonary disorder (COPD), rheumatoid arthritis, osteoarthritis, allergic disease, HIV/AIDS, asthma and cancer. Neuroinflammatory conditions include: multiple sclerosis (MS), Alzheimer's disease (AD) and Parkinson's disease (PD). A preferred method of the fifth aspect is the in vivo diagnosis or imaging of neuroinflammation. Most neurodegenerative diseases have an element of inflammation.

In a sixth aspect, the present invention provides the use of the pharmaceutical composition of the third aspect for imaging in vivo in a subject a CCR5 condition wherein said subject is previously administered with said pharmaceutical composition. The “CCR5 condition” and preferred embodiments thereof are as defined for the fifth aspect, above. By “previously administered” is meant that the step involving the clinician, wherein the imaging agent composition is given to the patient e.g. intravenous injection, has already been carried out.

In a seventh aspect, the present invention provides the use of the imaging agent of any one of the first aspect for the manufacture of a pharmaceutical for use in a method for the diagnosis of a CCR5 condition. The “CCR5 condition” and preferred embodiments thereof are as defined for the fifth aspect, above.

In an eighth aspect, the present invention provides a method of monitoring the effect of treatment of a human or animal body with a drug to combat a CCR5 condition, said method comprising administering to said body the pharmaceutical composition of the third aspect, and detecting the uptake of the imaging agent of said pharmaceutical composition. The “CCR5 condition” and preferred embodiments thereof are as defined for the fifth aspect, above.

In a ninth aspect, the present invention provides the pharmaceutical composition of the invention for use in a method for the diagnosis of a CCR5 condition. The “CCR5 condition” and preferred embodiments thereof are as defined for the fifth aspect, above.

The invention is illustrated by the following Examples.

Example 1 provides the synthesis of a non-radioactive ¹⁹F counterpart compound falling within Formula I of the present invention (“Compound 1”). Since the ¹⁸F version differs only in the fluorine isotope, it is chemically almost identical.

Example 2 provides the synthesis of a non-radioactive ¹⁹F counterpart compound falling within Formula II of the present invention (“Compound 8”). Since the ¹⁸F version differs only in the fluorine isotope, it is chemically almost identical.

Examples 3 and 4 provide prophetic examples of the syntheses of ¹⁸F-labelled Compounds 1 and 8. Example 5 provides a prophetic example of the syntheses of an ¹⁸F-labelled compound of Formula V.

Example 6 provides biological screening data for Compound 8 of Example 2. This shows that compound 8 binds CCR5 with high affinity and is selective for CCR5 as it does not bind CCR1 and CCR2B.

Example 7 provides the screening of Compounds 1 and 8 in a membrane permeability assay (PAMPA assay). Pe (permeability) predict a high CNS (blood brain barrier) permeability for Compound 8 and intermediate permeability for Compound 1.

Abbreviations.

The following abbreviations are used:

DCM=dichloromethane.

DIAD=diisopropyl azodicarboxylate.

DEA=diisopropylethylamine

DMF=N,N′-dimethylformamide.

EDCl=1-(3-dimethylaminopropyl)-3-ethylcarbodiimide hydrochloride.

HOBT=1-hydroxybenzotriazole.

LCMS=liquid chromatography mass spectroscopy.

THF=tetrahydrofuran.

### EXAMPLES

### Example 1

### Synthesis of Compound 1

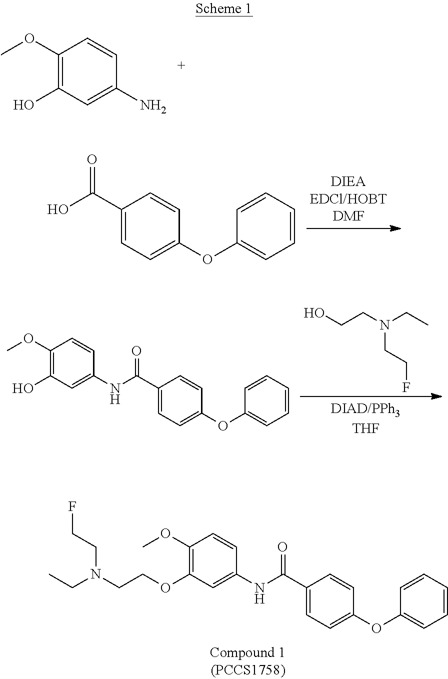

### Step (i): Synthesis of Compound A (Scheme 1)

A solution of 5-amino-2-methoxyphenol (2.78 g, 0.020 mol), 4-phenoxybenzoic acid (4.28 g, 0.020 mol), DIEA (3.1 g, 4.2 mL, 0.024 mol) and HOBT (3.2 g, 0.024 mol) in DMF (20 mL) was cooled to 0° C., EDCl (4.6 g, 0.024 mol) was added in one portion under nitrogen. The mixture was stirred at room temperature overnight. The mixture was poured into ice-water (100 mL) and extracted with ethyl acetate (50 mL×3). The combined ethyl acetate layer was washed with water, brine, dried (MgSO₄) and concentrated to dryness. The residue solid was triturated with DCM/hexanes, affording a white solid (4.1 g, 62%). ¹H NMR and LCMS analysis indicated _{>}98% purity.

### Step (ii): Synthesis of 2-(ethyl-2′-fluoroethylamino)ethanol

The required intermediate 2-(ethyl-2′-fluoroethylamino)ethanol was prepared as shown in Scheme 2

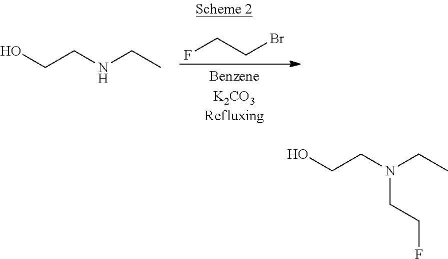

A mixture of 2-ethylaminoethanol (6.7 g, 0.075 mol), 2-fluoroethyl bromide (12 g, 0.094 moll, anhydrous potassium carbonate (10.3 g, 0.075 mol) and dry benzene (50 mL) was heated under reflux with stirring for 48 h. After cooling to room temperature, the solid was removed by filtration-and washed with benzene. The benzene was removed in vacuo. ¹H NMR analysis indicated the purity of the product was ˜90% and it was used directly in the next step without any further purification. (9.0 g, 89%).

### Step (iii): Synthesis of Compound 1

DIAD (1.36 g, 1.3 mL, 6.71 mmol) was added dropwise to a solution of Compound A from Step (i) (1.50 g, 4.47 mmol), 2-(ethyl-2′-fluoroethylamino)ethanol from Step (ii) (0.91 g, 6.71 mmol) and PPh₃ (1.76 g, 6.71 mmol) in anhydrous DCM (/THF (12 mL, 5:1). An exothermic reaction was immediately observed. The mixture was then stirred at room temperature overnight. The reaction mixture was concentrated to dryness and the residue solid was purified by column chromatography [silica, DCM→DCM/MeOH (98:2)]. The second fraction was the desired product, this fraction was chromatographed again under the same condition stated above. Recrystallisation from DCM/hexanes afforded a colourless solid (0.81 g, 40%). Analytical data indicated >98% purity. LCMS (API-ES+) m/z 453.3 (M+H⁺)

Supporting Data: ¹H NMR, ¹³C NMR, HPLC, LCMS of Compound 1.

### Example 2

### Synthesis of Compound 8

Compound 8 was prepared according to Schemes 3 and 4:

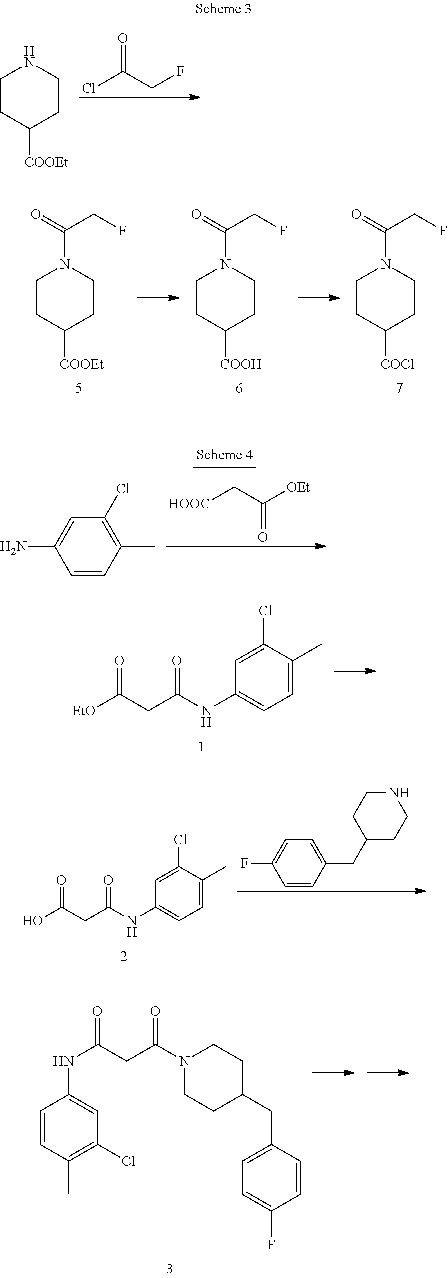

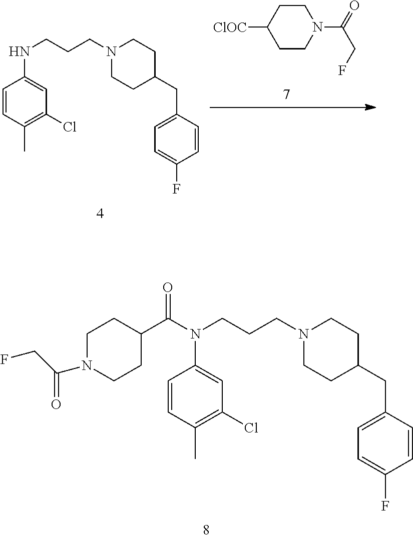

### Step (i): Synthesis of Compound 5

Ethylpiperidine-4-carboxylate (10 g, 0.064 mol, 1.1 eq) and triethylamine (15 ml, 2 eq) were dissolved in DCM (30 ml), and cooled on ice water bath with magnetic stirring. 2-Fluoroacetylchloride (5 g, 0.052 mol, 1.0 eq) in DCM (20 ml) was added dropwise (15 min) to the reaction and stirred for 1 h. Water (100 ml) was added and solvent was removed in vacuo. The residue was extracted into ethyl acetate (300 ml), which was washed with water, 2N HCl, saturated NaHCO₃, brine and dried over Mg₂SO₄. The organic solution was filtered, then concentrated. After silica gel column chromatography 6.2 g (yield 55%) of product compound 5 was obtained.

### Step (ii): Synthesis of Compound 6

Compound 5 (6.2 g, 0.029 mol, 1 eq) was dissolved in methanol (100 ml) and 2N NaOH (30 ml, 2 eq) was added and stirred overnight. The methanol was then removed in vacuo. The residue was acidified with 2N HCl to pH 3, extracted with ethyl acetate (3×500 ml), dried over MgSO₄, filtered and concentrated to afford compound 6 (4.3 g, yield 78%).

### Step (iii): Synthesis of Compound 7

Compound 6 (4.3 g, 0.023 mol) dissolved in DCM (50 ml), and DMF (one drop) was added, cooled on ice water bath, followed by addition of oxalyl dichloride (2.3 g, 1.5 ml, 2.5 eq) and stirred for 2 h. Solvent was removed and dry toluene (50 ml) was added to chase out possible residual solvent on a 50° C. water bath to give compound 7 (4.2 g, yield 88%).

### Step (iv): Synthesis of Compound 1 of Scheme 4

3-chloro-4-methylbenzenamine (15 g, 0.106 mol, 1 eq), 2-(ethoxycarbonyl)-acetic acid (14 g, 0.106 mol, 1 eq), diisopropylethylamine (16.5 g, 22.3 ml, 1.2 eq), HOBT (17 g, 1.2 eq) and DCM (150 ml) were mixed together. [1-ethyl-(3-dimethyl-amino-propyl)carbodiimide hydrochloride anhydrous] (EDCl, 24.5 g, 1.2 eq) was then added, and the resulting mixture stirred overnight under N₂. Workup, the reaction mixture washed with water, saturated NaHCO₃, 1N HCl and brine, dried over MgSO₄. Filtered off and concentrated to afford compound 1 (18.5 g, yield 70%).

### Step (v): Synthesis of Compound 2 of Scheme 4

Compound 1 from step (iv) above (18.5 g, ˜0.072 mol) was dissolved in methanol (100 ml), and cooled on an ice water bath. 2N NaOH (72 ml, 2 eq) was added and the mixture stirred overnight. Then solvent methanol was removed in vacuo. The residue was acidified with 2N HCl to pH 2 and extracted with ethyl acetate (500 ml), dried over MgSO₄, filtered off and concentrated to give compound 2 (13 g, 80%).

### Step (vi): Synthesis of Compound 3 of Scheme 4

Compound 2 from step (v) (4.9 g, ˜0.021 mol), 4-fluorobenzylpiperidine hydrochloride (4.9 g, 0.021 mol), diisopropylethylamine (12 g, 2.2 eq), HOBT (3.5 g, 1.2 eq) and DMF (150 ml) were mixed together. [1-ethyl-(3-dimethyl-aminopropyl)carbodiimide hydrochloride anhydrous] (EDCl, 5 g, 1.2 eq) was then added. The resulting mixture was stirred overnight under N₂. Workup, the reaction mixture was diluted with water (800 ml), extracted with ethyl acetate (500 ml), washed with water, saturated NaHCO₃, 1N HCl and brine, dried over MgSO4. Filtered off and concentrated to afford compound 3 (6.8 g, yield 80%).

### Step (vi): Synthesis of Compound 4 of Scheme 4

Compound 3 from step (v) (6.8 g, 0.017 mol) was dissolved in THF (100 ml), and BH₃ (1M solution in THF, 170 ml, 10 eq) was added and refluxed for 4 days till reaction complete (checked with LCMS). Solvent THF was then removed and methanol (100 ml) 6N HCl (100 ml) was added and refluxed for 5 days till reaction complete (checked with LCMS). Then methanol was removed and the residue was acidified to pH 11. Extracted with DCM (500 ml), dried over MgSO4, filtered off and concentrated (crude 6.2 g). After silica gel column give Compound 4 (3.7 g, 58%).

### Step (vii): Synthesis of Compound 8 of Scheme 4

Compound 4 from step (vi) (2.5 g, 0.0067 mol) and triethylamine(7 g, 10 ml, 0.068 mol, 10 eq) was dissolved in DCM (50 ml), cooled on ice water bath, added Compound 7 (4.2 g, ˜3 eq) in DCM (50 ml). The resulting mixture was stirred overnight. Reaction is messy but LCMS showed the desired product molecular weight. Workup, water was added, solvent DCM was removed in vacuo. Residue was extracted with ethyl acetate (500 ml), washed with water, saturated NaHCO₃, dried over MgSO₄, filtered off and concentrated, after silica gel column chromatography give final compound 8 (179 mg, 5%). LCMS (API-ES+) m/z 546.3 (M+H⁺).

### Example 3

### Synthesis of ¹⁸F-Labelled Compound 1 (Prophetic Example)

¹⁸F-labelled Compound 1 is prepared as shown in Scheme 5:

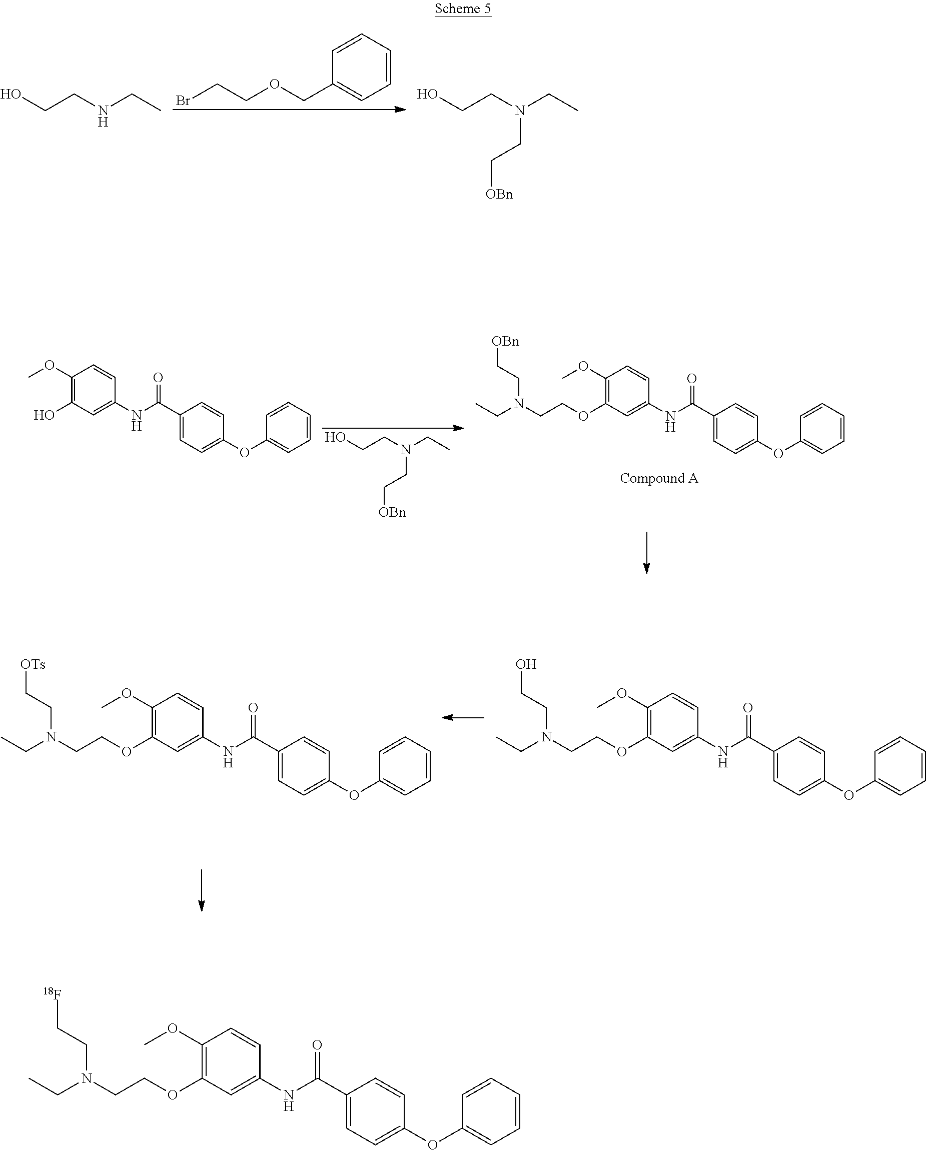

### Example 4

### Synthesis of ¹⁸F-Labelled Compound 8 (Prophetic Example)

The F-18 analogue of Compound 8 is synthesized from Compound 4 of Example 2 as shown in Scheme 6:

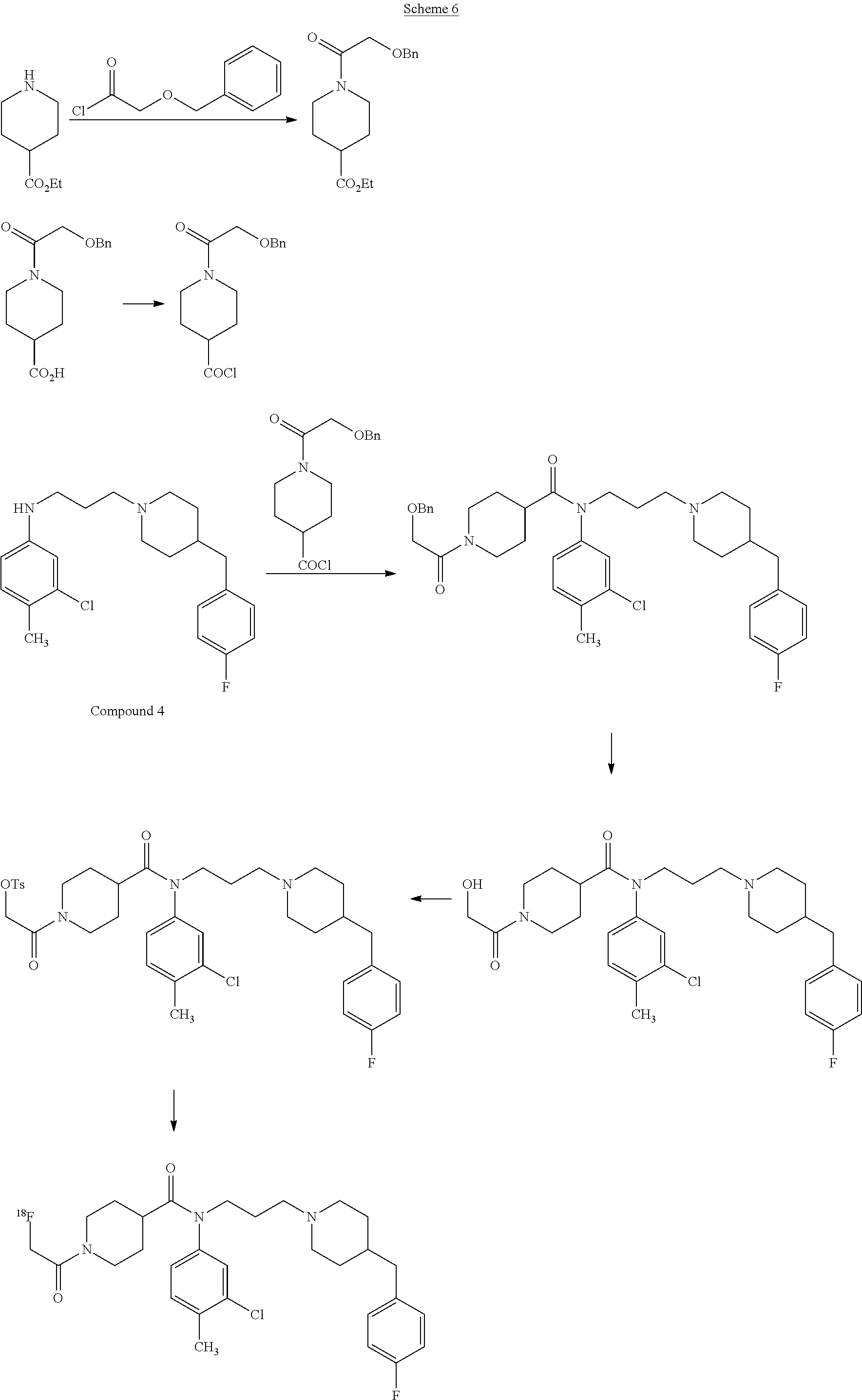

### Example 5

### Synthesis of ¹⁸F-Labelled Compound of Formula V (Prophetic Example)

An ¹⁸F-labelled Compound of Formula V is prepared as shown in Scheme 7:

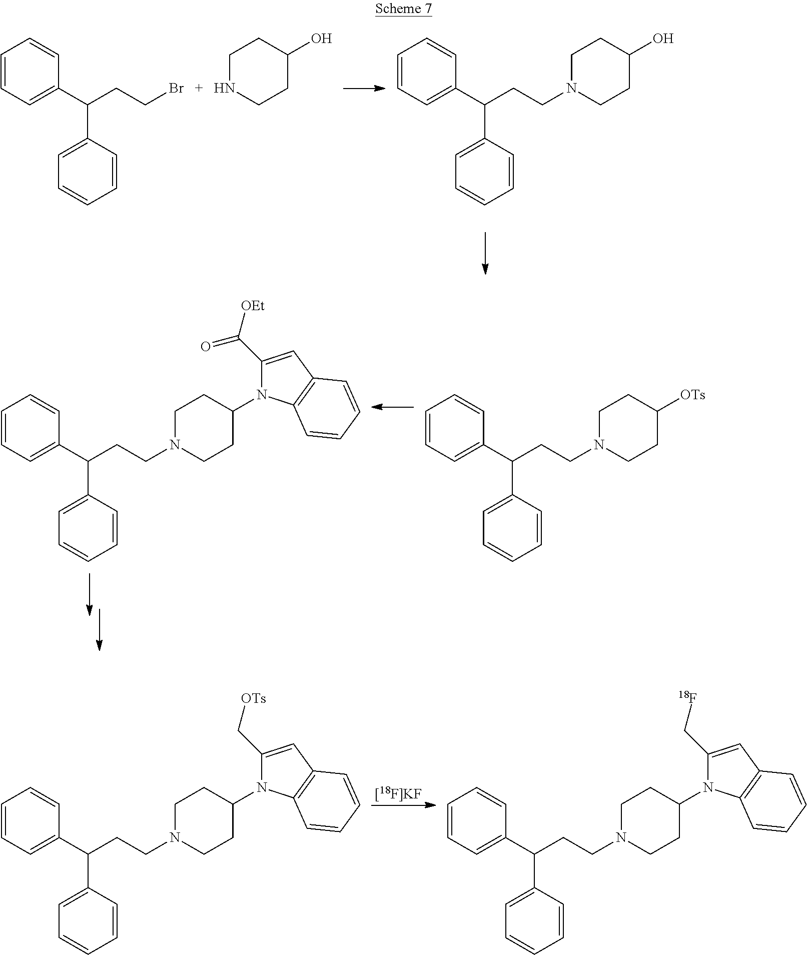

### Example 6

### Screening of Compound 8

Compound 8 of Example 2 was screened in CCR binding assays as follows:

The CCR1 binding assay was performed under the following conditions, according to a method that was adapted from the literature [Ben-Baruch et al., J. Biol. Chem., 270(38), 22123-8 (1995); Pease et al., J. Biol. Chem., 273(32), 19972-6 (1998)].

Thus, Compound 8 was incubated for 3 hours at 25° C. in 50 mM HEPES, pH7.4 containing 1 mM CaCl₂, 0.5% BSA, 5 mM MgCl₂ and 1% DMSO with Human recombinant CHO-K1 cells in the presence of 0.02 nM [¹²⁵I]-MIP-1α. MIP-1α is Macrophage Inflammatory Protein 1α (ligand of CCR1 and CCR5).

CCR2B binding assay was performed under the following conditions, according to a method that was adapted from the literature [Gong et al., J. Biol. Chem., 272, 11682-5 (1997); Moore et al., J. Leukoc. Biol., 62, 911-5 (1997)].

Thus, Compound 8 was incubated for 1. hour at 25° C. in 25 mM HEPES, pH7.4 containing 1 mM CaCl₂, 0.5% BSA, 5 mM MgCl₂, 0.1% NaN₃ and 1% DMSO with Human recombinant CHO-K1 cells in the presence of 0.1 nM [¹²⁵I]-MCP-1. MCP-1 is monocyte chemoattractant protein (the ligand of CCR2).

CCR5 binding assay was performed under the following conditions, according to a method that was adapted from the literature [Samson et al., J. Biol. Chem., 272, 24934-41 (1997)].

Thus, Compound 8 was incubated for 2 hours at 25° C. in 50 mM HEPES, pH7.4 containing 1 mM CaCl₂, 0.5% BSA, 5 mM MgCl₂ and 1% DMSO with Human recombinant CHO-K1 cells in the presence of 0.1 nM [¹²⁵I]-MIP-1β, where MIP-1β is Macrophage Inflammatory Protein 1β.

Compound 8 was found to be selective for CCR5 (Ki 0.79 nM) since binding affinity for CCR1 was much lower (32% binding inhibition at 10 μM Compound 8) and Compound 8 at 10 μM concentration did not inhibit the binding of MCP-1 to CCR2B.

### Example 7

### Permeability of Compound 8

The permeability of the CCR compounds was measured in a Parallel Artificial Membrane Permeability Assay (PAMPA) which gives a prediction of the blood brain barrier penetration by passive diffusion [Di et al, Eur. J. Med. Chem., 38(3), 223-232 (2003)].

The commonly accepted classification ranges for this PAMPA assay are as follows:
High predicted passive BBB permeation: Pe>4.0×10⁻⁰⁶ cm/sec.
Low predicted passive BBB permeation: Pe<2.0×10⁻⁰⁶ cm/sec.

Uncertain prediction of BBB permeation: 2.0×10⁻⁰⁶ cm/sec<Pe<4.0×10⁻⁰⁶ cm/sec.

The results were Pe=3.2E-06 cm/sec for Compound 1 and Pe=6.8E-06 cm/sec for Compound 8.

What is claimed is:

1-22. (canceled)

23) An imaging agent which comprises a synthetic compound having affinity for chemokine receptor 5 (CCR5) and having a molecular weight of 3000 Daltons or less, labelled with at least one imaging moiety, wherein following administration of said labelled compound to the mammalian body in vivo, the imaging moiety can be detected externally in a non-invasive manner and said imaging moiety is chosen from:
(i) a gamma-emitting radioactive halogen; or
(ii) a positron-emitting radioactive non-metal.)

24) The imaging agent of claim 23 where the synthetic compound is of Formula I: 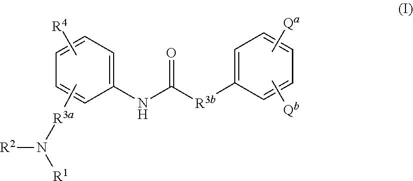
wherein:
R¹ and R² are independently C₁₋₆ alkyl, or C₁₋₆ haloalkyl;
R^{3a} and R^{3b} independently represent a bond, or a linker group selected from C₁₋₅ alkylene, —O—[C₁₋₄ alkylene]- or —[C₁₋₂ alkylene]-O—[C₁₋₂ alkylene]-;
R⁴ is selected from H, C₁₋₆ alkyl or C₁₋₆ alkoxy; and,
Q^{a} and Q^{b} are independently an A³ group or -(A²)ₙ-R⁵;
wherein A² is selected from —O—, —OCH₂—, —CH₂O—, CH₂, C═O, S═O, —NH(CO)— or —CO(NH)—, R⁵ is a phenyl group with 0-3 substituents which are A³ groups, and n is an integer of value 0 to 3;
wherein A³ is H, C₁₋₆ alkyl, OH or Hal.)

25) The imaging agent of claim 23 where the synthetic compound is of Formula II: 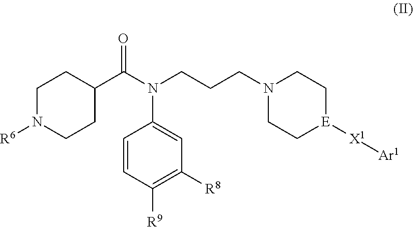
wherein:
R⁶ is acyl, fluoroacyl or methylsulfonyl; and,
R⁸-R⁹ are independently selected from H, C₁₋₃ alkyl, OH or Hal.
E is N or CH;
when E is N, X¹ is —CH₂— and when E is CH, X¹ is —CH₂— or —O—;
Ar¹ is a 6-membered aryl ring having 0-2 N heteroatoms, and substituted with 0 to 3 R⁷ groups;
each R⁷ is independently chosen from C₁₋₃ alkyl, OH, Hal, NO₂, NH₂, CO₂H, C₁₋₆ alkoxy, C₁₋₆ amino, C₁₋₆amido, —O(CH₂CH₂O)ₓX² or —NH(CH₂CH₂O)ₓX² where x is an integer of value 0 to 4, and X² is H or CH₃.)

26) The imaging agent of claim 23 wherein the synthetic compound is of Formula IIIa, Formula IIIb or Formula IIIc: 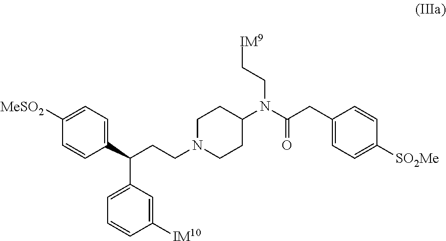
wherein:
IM⁹ and IM¹⁰ are independently H or an imaging moiety;
with the proviso that at least one of IM⁹⁻¹⁰ is an imaging moiety; 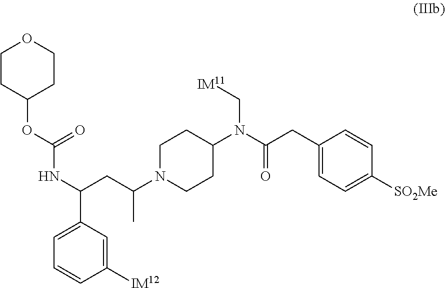
wherein:
IM¹¹ and IM¹² are independently H, CH₃ or an imaging moiety;
with the proviso that at least one of IM¹¹⁻¹² is an imaging moiety; 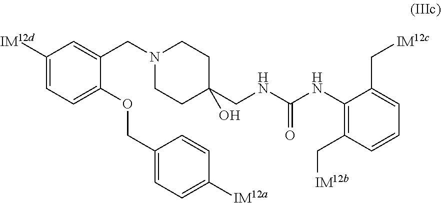
wherein:
IM^{12a}-IM^{12d} are independently H, CH₃ or an imaging moiety;
with the proviso that at least one of IM^{12a}-IM^{12d} is an imaging moiety.)

27) The imaging agent of claim 23 wherein the synthetic compound is of Formula IV: 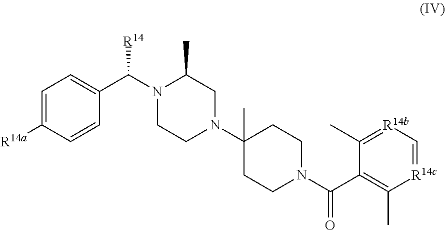
wherein R¹⁴ is H, C₁₋₆ alkyl, C₁₋₆ fluoroalkyl, C₁₋₆ alkoxy, or a phenyl or benzyl group optionally substituted with an A⁴ group;
wherein A⁴ is C₁₋₆ alkyl, C₁₋₆ alkoxy or Hal.
R^{14a} is selected from Hal or C₁₋₃ haloalkyl; and,
R^{14b} and R^{14c} are independently selected from CH or N.)

28) The imaging agent of claim 23 wherein the synthetic compound is of Formula V: 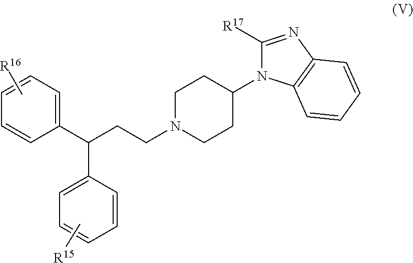
wherein:
R¹⁵ and R¹⁶ are independently H, OH, C₁₋₃ alkyl or Hal; and,
R¹⁷ is H, C₁₋₆ alkyl, or C₁₋₆ haloalkyl.)

29) The imaging agent of claim 23 wherein the synthetic compound is of Formula VI: 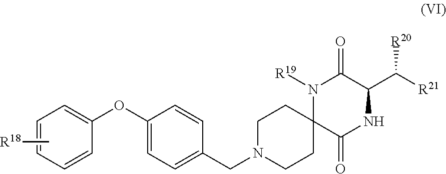
wherein:
R¹⁸ is H or Hal;
R¹⁹ is H, OH, or C₁₋₆ haloalkyl;
R²⁰ is H, OH or Hal; and,
R²¹ is C₁₋₆ alkyl, C₁₋₆ cycloalkyl, or C₁₋₆ haloalkyl.)

30) A method for the preparation of the imaging agent of claim 23, which comprises reaction of:
(i) a non-radioactive precursor; and,
(ii) a suitable source of the imaging moiety of claim 23,
wherein said precursor is a derivative of the synthetic compound of claim 23, and said derivative comprises a substituent Y¹ which is capable of reaction with said suitable source of the imaging moiety to give the imaging agent of claim 23.

31) A pharmaceutical composition which comprises the imaging agent of claim 23 together with a biocompatible carrier, in a form suitable for mammalian administration.)

32) The pharmaceutical composition of claim 31 for use in a method for the diagnosis of a CCR5 condition.)

33) A kit for the preparation of the pharmaceutical composition of claim 31 which comprises the precursor as defined in claim 30.

34) A method for the in vivo diagnosis or imaging in a subject of a CCR5 condition, comprising administration of the pharmaceutical composition of claim 31.

35) A method of monitoring the effect of treatment of a human or animal body with a drug to combat a CCR5 condition, said method comprising administering to said body the pharmaceutical composition of claim 31 and detecting the uptake of the imaging agent of said pharmaceutical composition.
